(19) United States
(12) Patent Application Publication
(10) Pub. No.: US 2022/0203114 A1
(43) Pub. Date: Jun. 30, 2022

(54) DOSIMETRY DETERMINATION FOR REGIONS WITHIN A TREATMENT AREA USING REAL-TIME SURFACE TEMPERATURE MAPPING AND ASSOCIATED METHODS

(71) Applicant: Accure Acne, Inc., Boulder, CO (US)

(72) Inventors: Henrik Hofvander, Budapest (HU); Michael John Estes, Lafayette, CO (US); Aubrey Jean Eck, Denver, CO (US)

(73) Assignee: Accure Acne, Inc., Boulder, CO (US)

(21) Appl. No.: 17/564,793

(22) Filed: Dec. 29, 2021

Related U.S. Application Data
(60) Provisional application No. 63/131,778, filed on Dec. 29, 2020.

(51) Int. Cl. A61N 5/067; A61N 5/06

(52) U.S. Cl. CPC A61N 5/067; A61N 2005/0628; A61N 5/0616

(57) ABSTRACT

An energy-based dermatological treatment system includes a temperature sensor for obtaining a first temperature measurement associated with a first treatment area. The system also includes a processing module for receiving the first temperature measurement and generate a temperature map based on the first temperature measurement. The system further includes a control module for setting a parameter of a first treatment pulse based on the first temperature map, and an energy source configured to deliver the first treatment pulse to the first treatment area. In an embodiment, the first temperature sensor is a contactless temperature sensor. In another embodiment, a second temperature measurement of a second treatment area to generate an updated temperature map based on the first and second temperature measurements. A parameter of a second treatment pulse is set according to the updated temperature map, and the second treatment pulse is delivered to the second treatment area.

### FIELD OF THE INVENTION

The present invention relates to energy-based treatments and, more specifically, systems and methods for determining and adjusting the dosimetry of a laser pulse based on a skin temperature map that can be generated and updated in real time to provide measured and estimated temperature data for a treatment area.

### BACKGROUND OF THE DISCLOSURE

Sebaceous glands and other chromophores embedded in a medium, such as the dermis, can be treated using thermal damage by heating the chromophore with a targeted light source, such as a laser. However, the application of enough thermal energy to damage the chromophore can also result in undesirable damage to the surrounding dermis and the overlying epidermis, thus leading to epidermis and dermis damage, as well as possible pain to the patient during treatment.

### SUMMARY OF THE DISCLOSURE

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its purpose is to present some concepts of one or more aspects in a simplified form as a prelude to the more detailed description to follow.

In an embodiment, an energy-based dermatological treatment system includes a temperature sensor for obtaining a first temperature measurement associated with a first treatment area, and a processing module for receiving the first temperature measurement and generate a temperature map based on the first temperature measurement. The system further includes a control module for setting a parameter of a first treatment pulse based on the temperature map, and an energy source for delivering the first treatment pulse to the first treatment area. In an embodiment, the parameter includes at least one of pulse intensity and pulse duration. In an embodiment, the temperature sensor is a contactless temperature sensor. In a further embodiment, the contactless temperature sensor includes an infrared sensor. In a still further embodiment, the energy source emits the first treatment pulse to the first treatment area, then a second temperature measurement of the first treatment area is taken to generate a second temperature map, and the control module is further configured to set at least one parameter of a second treatment pulse based on the second temperature map. In some embodiments, the system further includes a cooling unit for convecting heat from the first treatment area. The control module is operatively coupled with the cooling unit, and the control module is further configured for setting an operating parameter of the cooling unit based on the temperature map.

In another embodiment, a method of operating an energy-based dermatological treatment system including an energy source for delivering treatment pulses is disclosed. The method includes selecting a first treatment area, obtaining a first temperature measurement associated with the first treatment area, and generating a temperature map of the first treatment area based on the first temperature measurement. The method further includes setting a parameter of a first treatment pulse based on the temperature map, and delivering the first treatment pulse to the first treatment area. In an embodiment, the parameter includes at least one of pulse intensity and pulse duration. In a further embodiment, the method further includes defining a lower and/or an upper threshold value for the parameter, and generating an alert when the parameter is set below the lower threshold value or above the upper threshold value. In a still further embodiment, the method includes obtaining a second temperature measurement associated with the first treatment area, generating an updated temperature map of the first treatment area based on the first and second temperature measurements, adjusting a parameter of a second treatment pulse based on the updated temperature map, and delivering the second treatment pulse to the first treatment area. In an embodiment, the second temperature measurement is taken of a second treatment area, which may be adjacent to the first treatment area. The first and second treatment pulses may be delivered sequentially or substantially simultaneously. In an embodiment, the method further includes cooling the first and/or second treatment area prior to and/or during delivery of the first and/or second treatment pulses.

In another embodiment, a method for operating an energy-based dermatological treatment system is disclosed. The energy-based dermatological treatment system includes an energy source for delivering treatment pulses. The method includes selecting a first treatment area, delivering a first treatment pulse to the first treatment area, obtaining a first temperature measurement associated with the first treatment area, generating a temperature map of the first treatment area based on the first temperature measurement, and setting a parameter of a second treatment pulse based on the temperature map. In an embodiment, the method further includes cooling the first treatment area prior to delivering the first treatment pulse. In another embodiment, the method further includes delivering the second treatment pulse to the first treatment area or, alternatively, to a second treatment area. The second treatment area may be adjacent to the first treatment area. In still another embodiment, the second treatment is also cooled prior to and/or during delivery of the second treatment pulse.

### BRIEF DESCRIPTION OF THE DRAWINGS

The appended drawings illustrate only some implementation and are therefore not to be considered limiting of scope.

FIG. 1 shows a block diagram illustrating an energy-based photo-treatment system providing real-time skin temperature mapping and dosimetry feedback and adjustment capabilities, in accordance with an embodiment.

FIGS. 2A and 2B illustrate examples of simultaneous and sequential laser pulse application protocols, respectively, and associated temperature map generation procedures, in accordance with an embodiment.

FIG. 3 illustrates movement of a photo-treatment system to a different area of a patient's skin and generation of updated skin temperature maps, in accordance with an embodiment.

FIG. 4 shows a block diagram illustrating an energy-based photo-treatment system, including cooling and other functionalities, providing real-time dosimetry feedback, map-generation, and adjustment capabilities, in accordance with an embodiment.

FIGS. 5A and 5B show process flow diagrams describing methods of operating a photo-treatment system to obtain skin temperature measurements and generate a skin temperature map in real time, in accordance with an embodiment.

FIG. 6 illustrates a partial cutaway view of a portion of a scanner apparatus suitable for use with a photo-treatment system, in accordance with an embodiment.

FIG. 7 is a diagram illustrating a field of view (FoV) of a thermal sensor, in accordance with an embodiment.

FIG. 8 is a front view of a reference surface for use with a photo-treatment system, in accordance with an embodiment.

FIG. 9 is an isometric view of a reference surface, as viewed diagonally from the bottom, in accordance with an embodiment.

FIG. 10 is a process flow diagram illustrating an exemplary contactless method of sensing the temperature of the skin surface, in accordance with an embodiment.

### DETAILED DESCRIPTION

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof As used herein, the term “and/or” includes any and all combinations of one or more of the associated listed items, and may be abbreviated as “/”

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

In laser treatment of acne, the operating thermal range is generally bound on the upper end at the epidermis and dermis damage threshold temperature of approximately 55° C., and at the lower end by the temperature required to bring the sebaceous gland to its therapeutic damage threshold temperature of approximately 55° C. Based on clinical data, the operating temperature range for acne treatment expressed in terminal skin surface temperature is approximately 40° C. to 55° C., as an example. At skin surface temperatures below 40° C., it has been determined that there is no damage to the sebaceous gland, and thus no therapeutic benefit. When the skin surface temperature is between 40° C. and 55° C., there are varying degrees of sebaceous gland damage, with no epidermal damage. At skin surface temperatures above 55° C., there is dermal and epidermal damage in addition to effective therapeutic damage to the sebaceous gland.

The requirements for successful photo-thermal targeted treatment of specific chromophores with minimum patient discomfort include: 1) Epidermis sparing, namely making sure that the peak temperature value of the skin surface is less than around 55° C.; 2) Dermis sparing, namely avoiding overheating the dermis by balancing the average power of the treatment pulses with the heat extraction of the cooling system; and 3) Selective heating of the target chromophore, such as a peak temperature greater than 55° C. for sebaceous gland treatment. The embodiments described herein achieve the same effects as existing systems, with a much simpler system and protocol.

Tissue parameters, such as the thickness of the epidermis and dermis, vary among individuals, according to factors such as age, gender, and ethnicity, as well as between different skin locations. For example, the forehead has different tissue properties than the back, even for the same individual, thus necessitating different treatment parameter settings for the different treatment locations. Consideration of such variations in tissue properties in determining the specific treatment protocol is significant for laser-based treatments, e.g., the treatment of acne. Additionally, there may be variations in, for instance, the exact laser power, spot size, and cooling capacity between specific laser systems due to manufacturing variability and operating conditions.

Clinical data also indicates that terminal skin surface temperature has a strong dependence on tissue parameters at the specific treatment area for a particular individual. While existing treatment protocols have been based on a “one treatment fits all” type of an approach, an innovative analysis protocol can be incorporated into the treatment method so as to directly determine individually tailored treatment parameters extrapolating from measurements of terminal skin temperature at lower laser powers and/or the terminal skin surface temperatures reached during previous treatments, to avoid epidermis damage while efficaciously causing sebaceous gland damage. In this way, the treatment protocol can be customized for a specific treatment area for a particular individual, and also mitigates treatment variations that can be caused by variations in the laser power output of a specific machine, as well as variations in treatment conditions, such as ambient humidity and temperature.

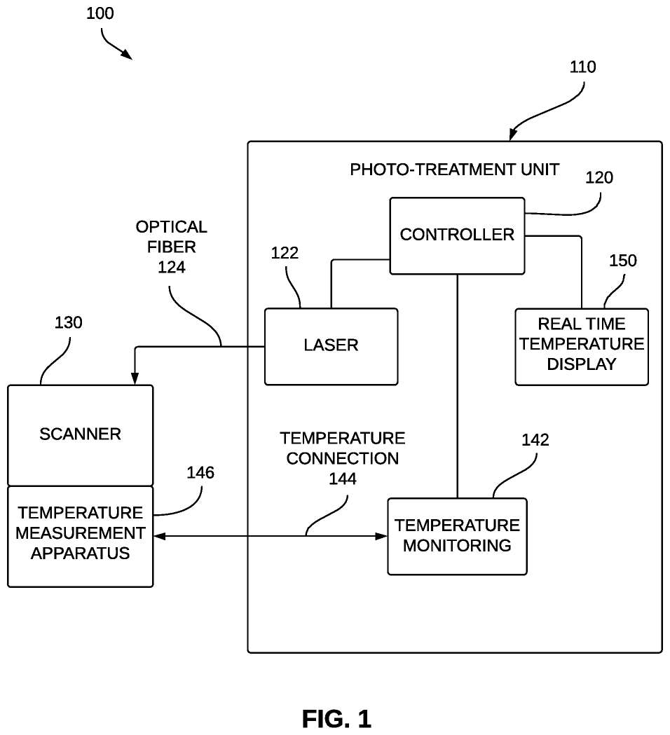

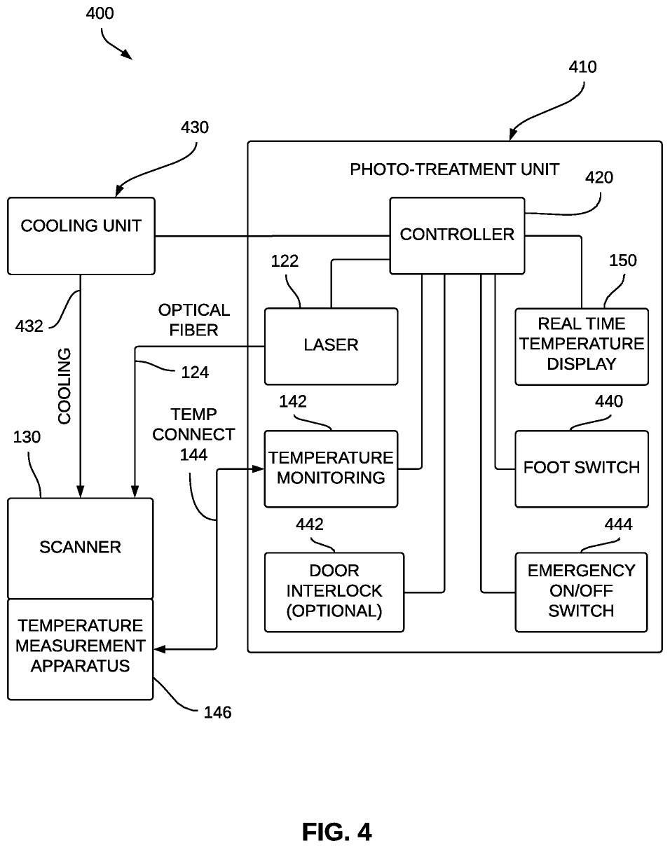

This analysis protocol can be performed by incorporating temperature measurements using, for example, a commercial, off-the-shelf, low-cost IR camera that can be built into the scanner (e.g., see temperature measurement apparatus 146 of FIGS. 1 and 4, as will be described below) that is held by the medical professional to apply the treatment to the patient, or by using a separate, commercial off-the-shelf single or multi-pixel thermal measurement devices. The prediction process can be performed on a highly localized level, thus adjusting the treatment protocol on the fly or prior to the treatment commencement, even adjusting the protocol for each individual region in the treatment area. In this way, the treatment protocol can be specified to provide the necessary treatment laser power while staying below the epidermis and dermis damage threshold temperature.

While there is not a good way to directly measure the temperature of the sebaceous gland being targeted by the treatment protocol, the skin surface temperature in the immediate area of the gland can be used as an indicator of the sebaceous gland temperature. A correlation model providing the correspondence between sebaceous gland temperature and skin surface temperature can then be used to tailor the actual treatment protocol using skin surface temperature measurements for effectively targeting sebaceous gland damage while staying below the damage threshold for the epidermis and dermis. The correlation model can be developed using, for example, an analytical heat transfer model, or by using clinical data (e.g., via biopsies) correlating skin surface temperature to sebaceous gland damage given the application of a specific treatment protocol.

A skin temperature map may be generated based on measured skin temperatures. Such a skin temperature map may be particularly beneficial when using treatment systems and protocols that deliver more than one laser pulse to a patient's skin in response to a single input provided by an operator (e.g., one “triggering” of the treatment device). The use of skin temperature maps and correlation models to adjust a laser pulse for specific region of skin within a treatment area will be described in detail herein below.

FIG. 1 shows a block diagram illustrating an energy-based photo-treatment system providing real-time dosimetry feedback and adjustment capabilities, in accordance with an embodiment. It is noted that the terms “photo-treatment,” “photo-thermal treatment,” and “energy-based dermatological treatment” are used interchangeably throughout the present disclosure, and all of these terms refer to the controlled delivery of energy (e.g., laser pulses) for treating dermatological conditions.

As shown in FIG. 1, a system 100 includes a photo-treatment unit 110, which in turn includes a controller 120 for controlling a laser 122. A laser power output from laser 122 is transmitted via an optical fiber 124 to a scanner 130. Scanner 130, for example, is a hand-held device used to apply the laser power output to the treatment location. Photo-treatment unit further includes a temperature monitoring unit 142 connected with scanner 130 via a temperature connection 144. For example, a temperature measurement apparatus 146, such as a thermistor, infrared camera, or other temperature sensing device is attached to or integrated into scanner 130 to provide real-time temperature measurement of the skin surface temperature at the treatment location. The temperature information measured by temperature measurement apparatus 146 may be transmitted via temperature connection 144 to temperature monitoring unit 142. Controller 120 may then transmit the temperature information to a real time temperature display 150, where the temperature information is viewable by a user of system 100.

Alternatively or additionally, the temperatures measured by temperature measurement apparatus 146 may be transmitted to controller 120, where a skin temperature map is generated based on the actual temperature measurements taken at known locations within the treatment area. Thermodynamic equations and/or empirical data may be used to approximate skin temperatures in portions of the treatment area that are not directly measured by the temperature measurement apparatus 146 in generating the skin temperature map. In some embodiments, to reduce reliance on estimations, equations, and empirical data, skin temperature may be measured in several places across the treatment area. For example, temperature measurements may be taken in regions that will receive a direct laser pulse from the photo-treatment system. In other embodiments, temperature measurements may be taken incrementally in a grid or other patterned arrangement. In still another embodiment, temperature measurement apparatus 146 may include an array of sensors, such as an infrared sensor array, and is thus capable of simultaneously obtaining temperature readings over a broad area of the treatment area, rather at single points. More skin temperature measurements may increase accuracy of the generated skin temperature map.

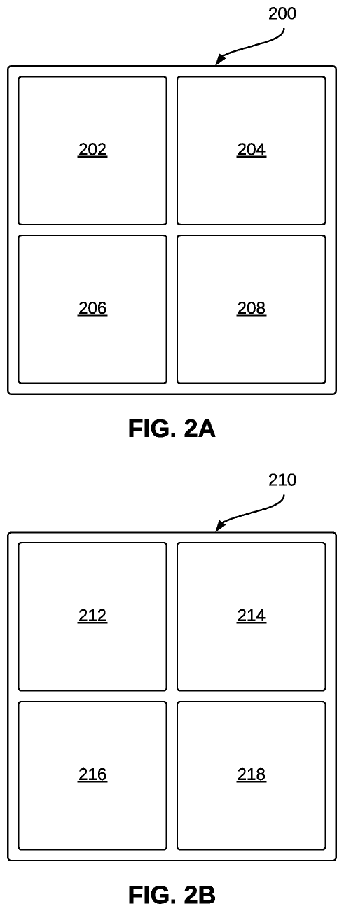

The skin temperature map may be particularly beneficial for treatment systems and protocols where multiple laser pulses are generated in response to a single operator input (e.g., a single trigger pull). FIGS. 2A and 2B illustrate two examples of such a protocol. Referring initially to FIG. 2A, a treatment area 200 is shown. Treatment area 200 represents an area that can be treated with a photo-treatment system, such as photo-treatment system 100, without repositioning the system. When the system is oriented toward a patient's skin, the treatment area 200 overlays the area of the patient's skin that will be treated. While treatment area 200 is shown as a square, the area can be an oval, circle, rectangle, or any other regular or irregular shape depending on the particular configuration of laser sources and/or shielding within the photo-treatment system.

Within treatment area 200 are regions 202-208. Each one of regions 202-208 represents a portion of the treatment area that can be affected by a single laser pulse from a photo-treatment system. In some embodiments, laser pulses directed at two or more of the regions 202-208 may be substantially simultaneously emitted by the photo-treatment system in response to a single input from an operator. While four square regions are shown, more or fewer regions of any shape may be present within treatment area 200 depending on the configuration of the associated photo-treatment system. The regions 202-208 may substantially abut each other such that there are no gaps in between the regions 202-208 and no areas of overlap. In some embodiments, each one of regions 202-208 may be approximately 5 mm by 5 mm in size; however, other sizes and grid arrangements (e.g., 1 by 1, 2 by 1, 3 by 3, 3 by 4, etc.) are possible.

As discussed above, skin temperatures can vary, even across a relatively small treatment area 200, which may be approximately 1 cm by 1 cm in size in some embodiments. Because the dermis and epidermis are sensitive to the laser pulses used to treat nearby sebaceous glands, it is desirable to project only the amount of energy needed to therapeutically heat the sebaceous glands within each one of regions 202-208 to a threshold terminal temperature (e.g., 55° C.) without heating the dermis and epidermis beyond the threshold terminal temperature. Tuning the laser pulses emitted by the photo-treatment system on a per region basis can help to reduce unwanted damage and discomfort to the patient. A cooling system may also be calibrated to pass cool air over the skin surface to extract heat.

An amount of adjustment of the laser pulse dose required for each region may be determined using the skin temperature map discussed above. In some embodiments, the skin temperature map may indicate that the skin temperature associated with region 202 is less than the skin temperature associated with region 204. Thus, for regions 202 and 204 to reach the same threshold terminal temperature, the dose of laser pulse delivered to region 202 may be higher than that delivered to region 204. For instance, the laser pulse delivered to region 202 may be at 100% intensity while the laser pulse delivered to region 204 may be at 97% intensity. As a further example, intensity and pulse duration of subsequent laser pulses delivered to region 202 may be adjusted quickly according to the measured skin temperature associated with region 204. The subsequent laser pulses may be delivered near simultaneously, or in close proximity in time and distance, such as in a time sequential manner. Similar adjustments may be made for regions 206, 208 based on the skin temperature map in combination with a correlation model that relates skin surface temperature to sebaceous gland, dermis, and/or epidermis temperature for a given area of skin. The correlation model may take into account where the treatment area is located on the body as well as age, gender, and ethnicity of the patient.

Referring to FIG. 2B, a second example treatment protocol is illustrated wherein the laser pulses are delivered to each region in a time sequential manner. Treatment area 210 includes regions 212-218 therein. While four square regions are shown, more or fewer regions of any shape may be present within treatment area 210 depending on the configuration of the associated photo-treatment system. The regions 212-218 may substantially abut each other such that there are no gaps in between the regions 212-218 and no area of overlap. The regions 212-218 may be approximately 5 mm by 5 mm in size; however, as discussed above with respect to regions 202-208, other sizes and configurations are possible without departing from the scope of the present disclosure.

In FIG. 2B, regions 212-218 receive a laser pulse at different times. In some embodiments, each region 212-218 within treatment area 200 receives a laser pulse at a unique time and each time sequential laser pulse is directed to a unique region within treatment area 210. Similar to the protocol of FIG. 2A, the laser pulse dose associated with each of regions 212-218 may be customized based on the skin temperature map and a correlation model. The photo-treatment system may automatically adjust the intensity and/or duration of the laser pulse delivered to each of the regions 212-218. Laser pulses in a time sequential configuration may be spaced apart by a time ranging from approximately 1 millisecond to approximately 1 second.

In some embodiments, additional mid-treatment temperature measurements are obtained for use in updating the skin temperature map throughout the photo-treatment in substantially real time. The mid-treatment temperature measurements may be taken in one or more of the same locations as the original temperature measurements and/or may be taken at other specific locations (e.g., near a region that will receive the subsequent laser pulse dose). Using these additional mid-treatment temperature measurements to update the skin temperature map in substantially real time may improve accuracy of the skin temperature map by taking into account thermal energy transferred to or from the skin during prior portions of the treatment. For example, when a first laser pulse is delivered to region 212, thermal energy may dissipate into adjacent regions 214, 216, thereby increasing skin temperatures in those regions. If this thermal crosstalk is not accounted for in adjusting subsequent laser pulse doses, laser pulses delivered to regions 214, 216 based on the initial skin temperature map alone may be too high and could result in terminal skin temperatures that are higher than the target temperature, thereby decreasing safety margins, damaging the dermis and epidermis, and/or causing pain to the patient.

In addition to adjusting the laser pulse dose, the treatment order of regions 212-218 may be adjusted such that as much space as possible is provided between one laser pulse and the subsequent laser pulse. For example, in FIG. 2B, it may be beneficial to deliver sequential laser pulses to region 212, region 218, region 214, region 216, etc. The treatment order and/or arrangement of the plurality of regions may be determined manually by the operator or may be automatically suggested or selected by a processor module, which may be local to or remotely coupled with, the photo-treatment system. The processor may also take into consideration the area of the body on which the treatment is performed and/or the age, gender, and ethnicity of the patient when recommending a specific treatment protocol.

A further variable that can be adjusted in the sequential treatment protocol is the time between laser pulses. Increasing the time between pulses may allow the skin to dissipate more heat and cool down to a temperature closer to the original skin temperature. However, over time, heat may also spread further into other treatment areas. The mid-treatment temperature measurements and real-time skin temperature mapping may assist with tracking the change in temperature over time and may provide information about when the next region within the treatment area 200 is ready to receive a laser pulse.

Other variables may also lead to skin temperature variations. For example, skin cooling processes, such as blowing cool air over the surface of the skin, can be performed during the treatment protocol to convect heat away from the skin and prevent overheating of the epidermis and dermis. Variations in air flow patterns, air temperature, humidity, and other cooling variables may cause heat to be extracted from the skin non-uniformly, thereby leaving warmer and cooler spots within the treatment area. As discussed above, inability to account for a warm area may lead to surrounding tissue damage due to overheating. Inability to account for a cooler area may reduce efficacy of the photo-treatment if the underlying sebaceous gland in not heated to the threshold terminal temperature. Thus, it is beneficial to identify substantially real-time skin temperature using skin temperature mapping that continuously updates to accurately represent skin temperature as additional measurements are collected. Adjustments to increase or decrease laser pulse dosage based on the real-time skin temperature map can be made manually by the operator and/or can be suggested or selected automatically by the photo-treatment system.

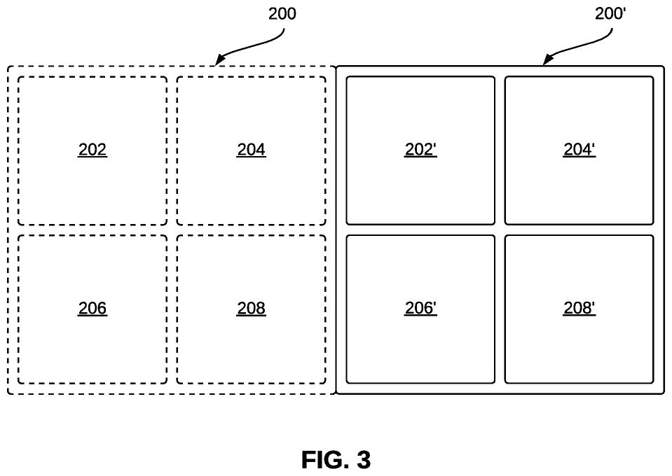

For both the simultaneous treatment protocol described with respect to FIG. 2A and the sequential treatment protocol described with respect to FIG. 2B, the photo-treatment system may be repositioned over a different portion of the patient's skin to continue treatment over a larger portion of the patient's skin than the treatment area 200 can reach. An example is shown in FIG. 3 where the treatment area 200′ represents the photo-treatment device moved to a second position to the right of the previously treated area 200. Depending on the distance between first treatment area 200 and the second treatment area 200′, thermal crosstalk may occur between one or more previously treated regions 202-208 and one or more of the regions 202′-210′ yet to be treated. Thus, a continuation of real-time skin temperature measurement and skin temperature mapping over an area larger than the immediate treatment area may be beneficial for taking into account prior thermal changes in nearby skin that could affect subsequent stages of the treatment. When the updated and extended skin temperature map is generated and data is available for determining the next set of laser pulses for the regions 202′-210′ (whether simultaneous or sequential), the operator may receive a prompt from the photo-treatment system indicating that a treatment protocol for the treatment area 200′ is ready. In other embodiments, the operator may receive a prompt indicating that treatment area 200′ overlaps previous treatment area 200 and the photo-treatment system position should be adjusted to prevent overtreating the overlap area.

A trigger pull by the operator may initiate treatment including multiple laser pulses delivered to one or more of the regions 202′-210′ as discussed above with respect to FIGS. 2A and 2B. Because of the ever-changing nature of skin temperature during the photo-treatment process, it may be beneficial to deliver the laser pulse as soon as possible after the real-time skin temperature map is updated and the pulse dose is determined. For example, it may be desirable to deliver the laser pulse within 10 milliseconds of determining the dose for the selected region.

FIG. 4 shows a block diagram of an energy-based photo-treatment system, including cooling and other functionalities, providing real-time dosimetry feedback, real-time skin temperature measurement and mapping, and adjustment capabilities, in accordance with an embodiment.

A system 400 includes the components from system 100 of FIG. 1, including laser 122, optical fiber 124 transmitting a laser power output to scanner 130, temperature monitoring unit 142, temperature connection 144, temperature measurement apparatus 146 attached to or integrated into scanner 130, and a real time temperature display 150. A photo-treatment unit 410, containing several of these components, also includes a controller 420, which is configured for controlling the operations of laser 122, temperature monitoring unit 142, real time temperature display 150, foot switch 440, optional door interlock 442, and emergency on/off switch 444. System 400 also includes additional components (required and optional), including a cooling unit 430 and a cooling connection 432. Additional examples and experimental results relating to the system of FIGS. 1 and 4 are described in U.S. Provisional Patent Application No. 62/824,995 filed on March 27, 2019.

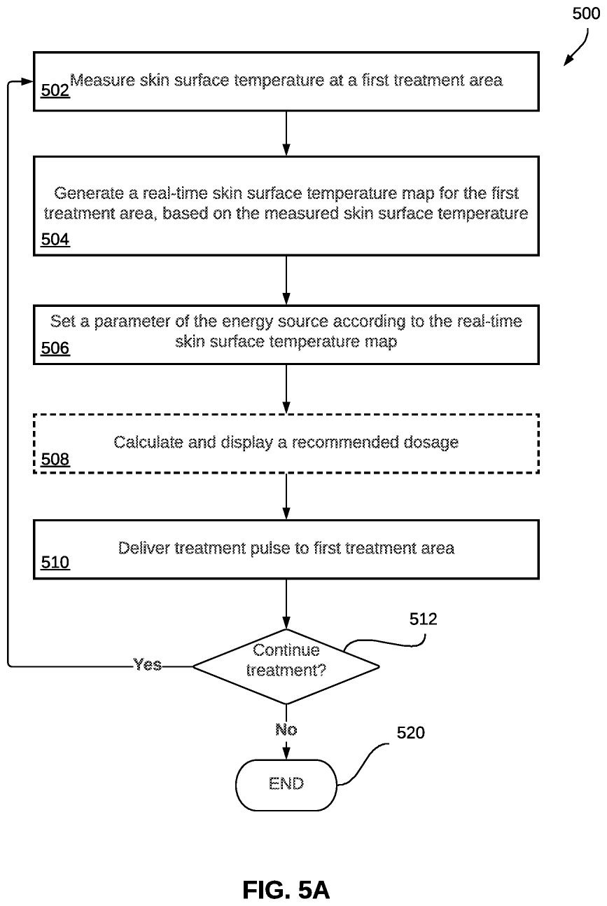

FIGS. 5A and 5B show flow charts illustrating methods of operating an energy-based dermatological treatment system incorporating real-time measurement and mapping of the skin surface temperature. Referring first to FIG. 5A, a treatment method 500 uses an energy-based photo-treatment system incorporating an energy source (e.g., a laser) such as those shown in FIGS. 1 and 4, in accordance with an embodiment.

As shown in FIG. 5A, a treatment method 500 begins by measuring the skin surface temperature at a first treatment area in a step 502. The temperature measurement of step 502 may include, for instance, the sequential measurement of temperature at a variety of points within the first treatment area or the simultaneous measurement of temperatures over an area, such as using an array sensor or an infrared camera. Then, in a step 504, a temperature map for the first treatment area is generated, based on the measured skin surface temperature from step 502. In an embodiment, the temperature map is generated to indicate the skin surface temperature over the first treatment area essentially in real-time, incorporating the most recently measured skin surface temperature measurements.

The temperature map is then used to set a parameter of the energy source in a step 506. The parameter may include, for instance, the intensity or the duration of one or more of the energy (e.g., laser pulses) to be delivered by the energy source at the first treatment area. As an example, if the first treatment area has been sufficiently cooled using a cooling unit (e.g., cooling unit 430 in FIG. 4), then the patient being treatment may be able to tolerate a higher energy laser pulse at the first treatment area.

Optionally, treatment method 500 may include a step 508 to calculate and display a recommended dosage (i.e., settings of the energy source parameters) to the user of the treatment system. Assuming a knowledgeable user experienced with the various settings options and pain thresholds of the patient being treated, the user may opt to make further adjustments in the treatment protocol, such as to increase the cooling provided by the cooling unit or to terminate the treatment.

Treatment method 500 then proceeds to a step 510 to deliver the treatment pulse (or multiple pulses) to the first treatment area with the adjusted energy source parameters. A decision 512 is then made whether to continue the treatment. Decision 512 may be based, for example, on the reaction of the patient to the treatment pulse delivered in step 510, visual observation of the condition of the skin surface at the first treatment area, or another skin surface temperature measurement. If the answer to decision 512 is YES, then treatment method 500 returns to step 502 to take another set of skin surface temperatures and to update the temperature map. In the additional iterations of treatment method 500, the steps may be performed again at the first treatment area, or at another treatment area (adjacent to the first treatment area or remote from the first treatment area). If the answer to decision 512 is NO, then the treatment is ended in a termination step 520.

Turning now to FIG. 5B, an alternative treatment method, in accordance with an embodiment, is illustrated. As shown in FIG. 5B, a treatment method 550 begins by delivering one or more treatment pulses at an initial setting of the energy source for a first treatment area in a step 552. For example, the initial setting of the energy source may be set intentionally lower than the known damage threshold of the dermis and epidermis, or much lower than the energy settings known to cause a painful sensation to the patient.

In a step 554, the skin surface temperature is measured in at least one location with the first treatment area, then temperature map for the first treatment area is generated in a step 556, based on the measured skin surface temperature. As described above with respect to FIG. 5A, the temperature map may be generated in essentially real time, using the latest known skin surface temperature information for the first treatment area.

From the temperature map, a recommended dosage for one or more additional treatment pulses is generated in a step 558. The recommended dosage may include, for example, various parameter settings for the energy source, such as the laser treatment pulse intensity, pulse duration, duty cycle, etc., or a temperature setting which may be translated by a system controller into specific parameter settings for the energy source. Optionally, the recommended dosage is displayed in a step 560 for viewing by a user.

Then, a determination is made whether the initial laser settings for the first set of treatment pulses were too high or too low in a step 562. This determination may be made by the user of the treatment system based on the recommended dosage display in step 560, patient reaction to the initial treatment pulse delivery, visual inspection of the first treatment area, or other factors. Alternatively, determination 562 may be made automatically by the treatment system according to preset lower and/or upper thresholds for the measured skin temperature and/or energy source parameter settings. For instance, the treatment system may include preset thresholds such that the user cannot accidentally deliver laser pulses with energy higher than known pain tolerances. Optionally, the treatment system may include override sequences to be able to set the energy source parameters above or below the preset system thresholds, in order to provide the user with additional flexibility in the customization of the treatment protocol.

If determination 562 concludes the initial parameter settings of the energy source were too high or too low, then a determination 564 is made whether to adjust the parameter settings (e.g., power settings of the laser). If determination 564 further concludes an adjustment in the energy source parameter settings (e.g., laser power) is required, then the necessary adjustments are made in a step 566. A determination 568 is then made whether or not to continue treatment. If determination 568 concludes additional treatment is necessary, then treatment method 550 returns to step 552. If no further treatment is deemed to be necessary, treatment method 550 concludes in a termination step 570. If determination 562 concludes the initial parameter settings were adequate, or if determination 564 concludes no parameter adjustment is necessary, treatment process 550 also proceeds to determination 568. Upon return to step 552, treatment method 550 may be repeated for the first treatment area, or be applied to a second treatment area adjacent or remote from the first treatment area.

The processes described in FIGS. 5A and 5B are examples of a process control, which combines a process of measuring the skin surface temperature and generating a skin temperature map in real time with a control strategy based on the relationship between, for instance, laser power and skin surface temperature, with the optional control action to increase or decrease laser power (or other parameter settings for the energy source). Additionally, if a cooling mechanism, such as cooling unit 430 in FIG. 4, is provided within the energy-based treatment system, one or more parameters of the cooling unit, such as the air flow rate and air temperature, can also be adjusted based on the measured or estimated (based on the temperature map) skin surface temperature. The adjustment of the energy source and/or cooling unit parameters can be performed manually by a user, or automatically by a controller unit, such as controller 120 of FIG. 1 or 420 in FIG. 4. Additionally, the adjustment of the energy source and/or cooling unit can be performed repeatedly and continuously during the treatment protocol such that the desired skin surface temperature is maintained, regardless of variations in the treatment location characteristics, energy source output, and cooling unit output. Further, it is noted that the temperature map generation may be performed prior to or following the application of the initial treatment pulses.

The mapping and correlation models described above increase the effectiveness and safety of the treatment when they are predicated on accurate skin surface temperature measurements. There are various contactless methods of measuring skin surface temperature during, for example, dermatological procedures. Devices such as infrared (IR) cameras, pyrometers, bolometers, and dual-wavelength sensors can provide a reading of the skin surface temperature. However, for procedures such as photo-thermal targeted treatment to cause thermal damage to subcutaneous sebaceous glands, accurate, calibrated reading of the skin surface temperature can prevent damage to the epidermis and dermis in and around the treatment area.

The system and associated methods described in U.S. Provisional Patent Application No. 62/804,719 and PCT Patent Application No. PCT/US20/12473, both of which applications are incorporated herein by reference in their entirety, provide a fast, inexpensive, and compact system and method to significantly improve the accuracy of contactless temperature measurements. The inclusion of such an accurate, real-time temperature measurements of the treatment area enables new energy-based treatment systems that allow real-time, on-the-fly adjustment of the treatment dosimetry that was not previously possible. Additionally, a visual display (e.g., real time temperature display 150 of FIG. 1 or 4) of the real-time temperature measurements provides the user of the system with feedback that can be used as the user controls the energy output of the photo-treatment system, or the output of the cooling system, or both, thus leading to increased user satisfaction, enhanced safety, and improved efficacy. The measurement system may further transmit the real-time temperature measurement data corresponding to a plurality of points within a treatment area to a processing module, which may be local or remote relative to the treatment system, for generating a real time skin temperature map. The accurate measurement system in combination with the processing module may calculate or otherwise define safe operating ranges for the parameters of the light source and the cooling source which will achieve a desired skin surface temperature. The desired skin surface temperature may be chosen such that unwanted thermal damage at the location to be treated is avoided yet the treatment is effective.

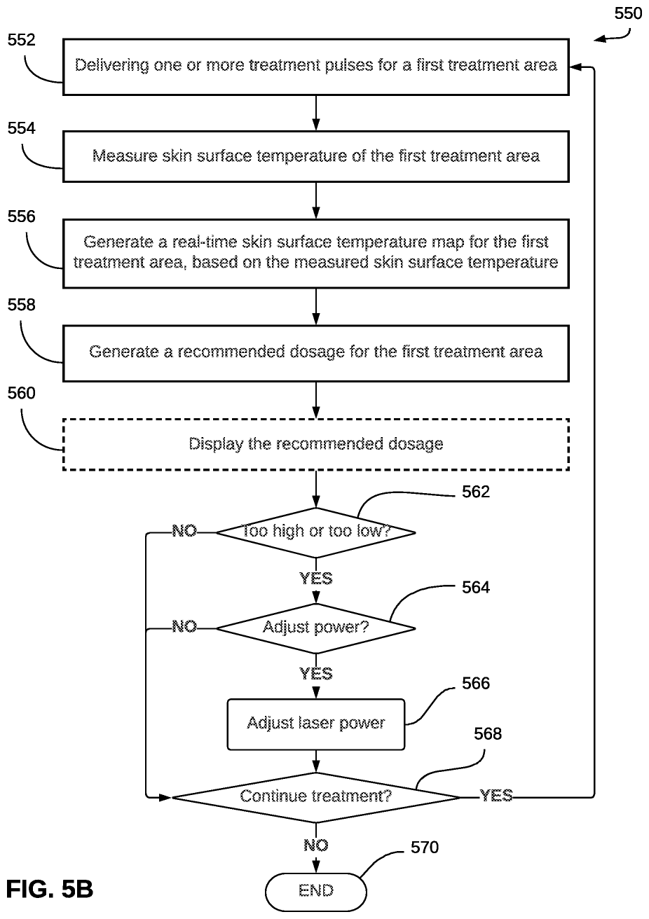

FIG. 6 illustrates a side view of a portion of a scanner apparatus suitable for use with the photo-thermal treatment system 100, in accordance with an embodiment. A scanner 600 includes an optical fiber 602 for transmitting a laser beam 604 from a base station (not shown) along a laser beam path 610 toward a treatment tip 620, which is placed in contact with the treatment location. Scanner 600 can optionally include optical components for shaping the light beam projected onto the skin at treatment tip 620.

Treatment tip 620 serves as a visual guide for the user to position scanner 600 at a desired treatment location. In order to allow contactless temperature measurement, an IR camera 630 is attached to scanner 600 and points downward toward treatment tip 620 such that IR camera 630 is able to detect the temperature of the treatment location along an optical path 635. In an embodiment, IR camera 630 has a fast time response, for example less than 40 milliseconds, between consecutive surface temperature measurements. Additionally, in the embodiment shown in FIG. 6, scanner 600 includes a cooling air duct 640. As an example, an air hose (not shown) can be attached to cooling air duct 640 via a threaded opening 642. Alternate configurations of the scanner device can include one or more scanning optical components configured to redirect laser beam path 610 and/or optical path 635 in one or two dimensions to provide additional degrees of freedom for laser pulse delivery and IR temperature measurement.

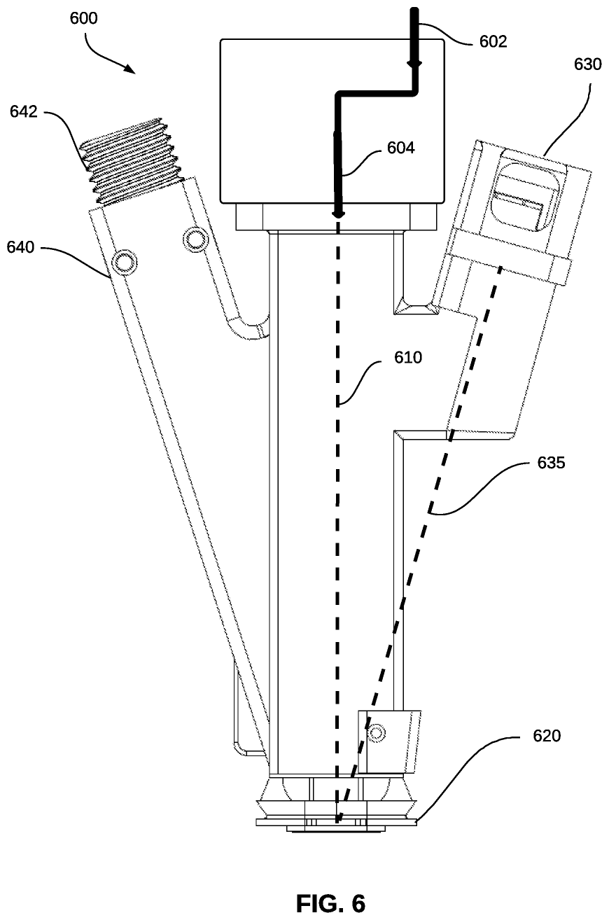

FIG. 7 illustrates a field of view (FoV) of IR camera 630 looking toward treatment tip 620. FoV 710 of the IR camera is represented by an oval, in accordance with an embodiment. Visible within FoV 710 are treatment tip 620 and a reference surface 730, attached to an inner surface of scanner 600. Thus, IR camera 630 is capable of simultaneously measuring the temperature of skin within a treatment area and reference surface 730.

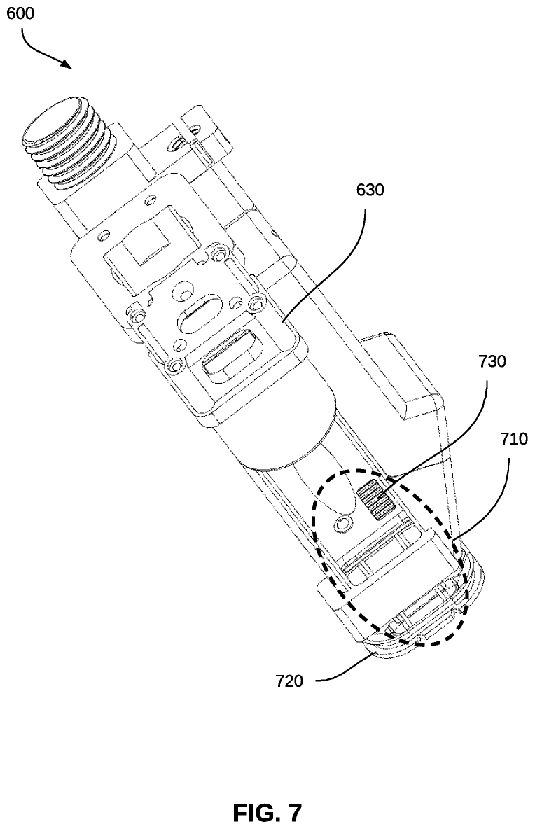

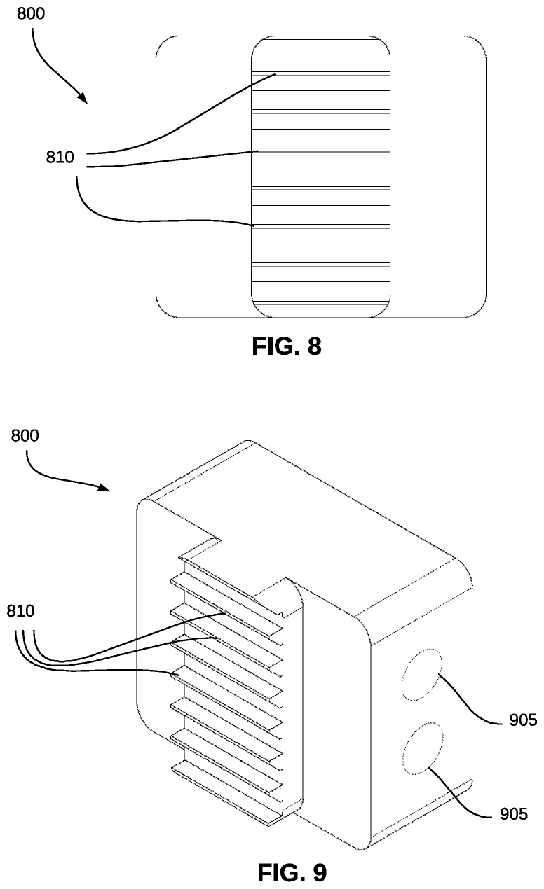

Further details of the reference surface, in accordance with an embodiment, are illustrated in FIGS. 8 and 9. FIG. 8 is a front view of a reference surface and FIG. 9 is an isometric view of the reference surface, as viewed diagonally from the bottom, in accordance with an embodiment. As shown in FIGS. 8 and 9, a front surface of reference surface 800 includes a texture 810, which steers reflections and stray light from any surface other, than the reference surface itself, away from FoV 710. In an exemplary embodiment, reference surface 800 also includes one or more mounting holes (not shown) through which reference surface 800 can be attached to, for example, an inside surface of scanner 600 as shown in FIG. 6. Alternatively, reference surface 800 is captively attached or otherwise mounted onto an appropriate location within the FoV of the IR camera. In an embodiment, the reference surface is characterized by a reference emissivity value approximately equal to a measured emissivity value of the measured skin surface. In another example, a surface coating on the reference surface exhibits a light scattering property that is approximately Lambertian, not specular. Further details relating to configurations of the reference surface are described in U.S. patent application Ser. No. 16/734,280, filed Jan. 3, 2020.

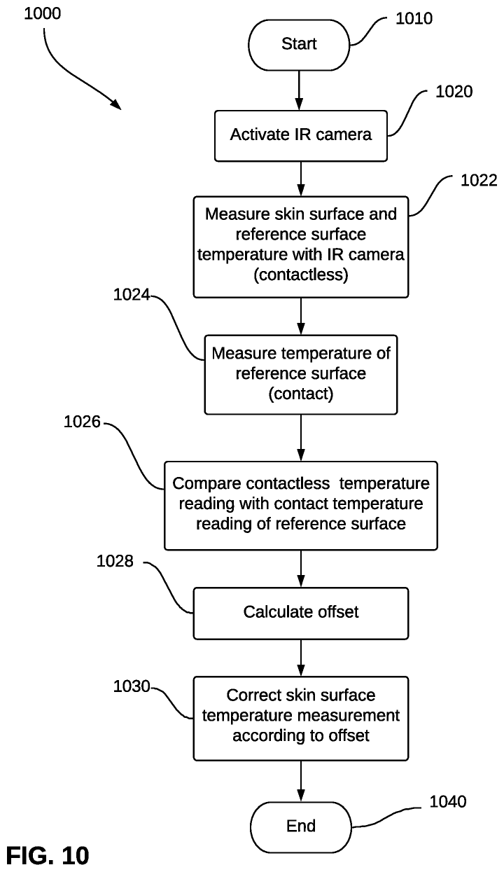

FIG. 10 is a flow diagram illustrating an exemplary contactless method of sensing the temperature of the skin surface, in accordance with an embodiment. As shown in FIG. 10, a process 1000 begins with a start step 1010, in which the temperature sensing protocol is activated. Then, in a step 1020, an IR camera in a setup such as shown in FIG. 6 is activated. IR camera then measures the skin surface temperature and the reference surface temperature in a step 1022. Some IR cameras have an internal self-correction/calibration/shutter mechanism. One such self-correction is a so-called “flat field correction,” which ensures that each pixel in the camera measures the same temperature of a constant-temperature surface. The method described in FIG. 10 uses a reference surface that is provided externally to the IR camera. In parallel, a temperature reading of the reference surface is taken with the contact sensor within the reference surface in a step 1024. In a step 1026, the reference surface temperature taken by the IR camera in step 1022 is compared with the temperature reading of the reference surface taken with the contact sensor within the reference surface in step 1024. An offset, if any, between the temperature measured in step 1022 and the reading taken in step 1024 is calculated in a step 1028. In a step 1030, the offset calculated in 1028 is used to correct the skin surface temperature measurement taken by the IR camera. Process 1000 is ended in an end step 1040.

In other words, by comparing the reference surface temperature, as measured by the contact-less sensor, with a known, high accuracy contact measurement taken of the same reference surface, an offset is calculated, which is used to correct the temperature reading of the skin surface. As a result, the accuracy of the contact-less measurement is greatly improved, regardless of the specific treatment protocol, skin cooling procedures, patient parameters (e.g., age, gender, ethnicity, specific treatment location). It is noted that the contact temperature measurement taken in step 1024 of process 1000 does not need to occur with every contactless temperature measurement taken in 1022. For example, after the offset has been calculated once, steps 1024, 1026, 1028, and 1030 can be performed periodically to correct for potential calibration errors. This method of contactless temperature measurement is particularly relevant for temperature mapping as, by using an infrared camera with numerous pixels, for example, the accuracy of the temperature mapping may be improved over contact temperature measurements.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, many different embodiments stem from the above description and the drawings. It will be understood that it would be unduly repetitious and obfuscating to literally describe and illustrate every combination and subcombinations of these embodiments. As such, the present specification, including the drawings, shall be construed to constitute a complete written description of all combinations and subcombinations of the embodiments described herein, and of the manner and process of making and using them, and shall support claims to any such combination or subcombination.

For example, embodiments such as the items below are contemplated:

1. An energy-based dermatological treatment system including:
a temperature sensor configured to obtain a first temperature measurement associated with a first treatment area;
a processing module configured to receive the first temperature measurement and generate a temperature map based on the first temperature measurement;
an energy source configured to emit a first treatment pulse to the first treatment area; and
a control module configured to set at least one parameter of the first treatment pulse based on the first temperature map,
wherein first temperature sensor is a contactless temperature sensor.

2. The system of Item 1, wherein the contactless temperature sensor includes an infrared temperature sensor.

3. The system of Item 1, wherein the at least one parameter includes at least one selected from the group consisting of pulse intensity and pulse duration.

4. The system of Item 1, wherein the temperature sensor is further configured to obtain a second temperature measurement associated with a second treatment area, wherein the processing module is configured to generate an updated temperature map based on the first and second temperature measurements, and wherein the energy source is further configured to emit a second treatment pulse to the second treatment area.

5. The system of Item 4, wherein the control module is configured to set at least one parameter of the second treatment pulse based on the updated temperature map.

6. The system of Item 4, wherein the control module is configured to set at least one parameter of the first and second treatment pulses based on the first and second temperature measurements.

7. The system of Item 1, further comprising a cooling unit configured to convect heat from the first treatment area.

8. The system of Item 7, wherein the control module is operatively coupled with the cooling unit and wherein the control module is configured to adjust at least one operating parameter of the cooling unit based on the temperature map.

9. A method of operating an energy-based dermatological treatment system including:
aiming the energy-based dermatological treatment system at a first region of a first treatment area;
obtaining a first temperature measurement associated with the first region of the first treatment area;
generating a temperature map of the first treatment area based at least in part on the first temperature measurement;
setting at least one parameter of a first treatment pulse based on the temperature map; and
selectively emitting from an energy source the first treatment pulse to the first region of the first treatment area.

10. The method of Item 9, further including obtaining a second temperature measurement associated with a second treatment area.

11. The method of Item 9, further including generating a first updated temperature map of the first treatment area based at least in part on the first and second temperature measurements.

12. The method of Item 11, wherein setting the at least one parameter of the first treatment pulse is based on the first updated temperature map.

13. The method of Item 12, wherein the at least one parameter comprises one selected from the group consisting of pulse intensity and pulse duration.

14. The method of Item 12, further including selectively emitting from the energy source a second treatment pulse to the second treatment area.

15. The method of Item 14, wherein the first and second treatment pulses are emitted sequentially.

16. The method of Item 15, wherein the first and second treatment pulses have the same parameters and are emitted substantially simultaneously.

17. The method of Item 9, further including:
aiming the energy-based dermatological treatment system at a third treatment area;
obtaining a third temperature measurement associated with the third treatment area;
generating a second updated temperature map of the first and second treatment areas based at least in part on the first, second, and third temperature measurements;
setting at least one parameter of a third treatment pulse based on the second updated temperature map; and
selectively emitting from an energy source the third treatment pulse to the third treatment area.

18. The method of Item 17, further including generating an alert when the second treatment area overlaps the first treatment area.

19. The method of Item 17, further including generating an alert when the at least one parameter of a third treatment pulse is set at a value below an effective treatment value.

20. The method of Item 9, further including cooling at least the first treatment area with a cooling unit configured to convect heat away from the first treatment area.

21. The method of Item 9, wherein obtaining the first temperature measurement comprises measuring the first region within the first treatment area using a contactless temperature sensor.

22. The method of Item 21, wherein the contactless temperature sensor comprises an infrared sensor.

23. An energy-based dermatological treatment system comprising:
an energy source configured to emit a first treatment pulse to a first treatment area;
a temperature sensor configured to obtain a first temperature measurement associated with the first treatment area;
a processing module configured to receive the first temperature measurement and generate a temperature map of the first treatment area based on the first temperature measurement;
a control module configured to adjust at least one parameter of a second treatment pulse to be emitted by the energy source based on the first temperature map,
wherein first temperature sensor is a contactless temperature sensor.

24. The system of Item 23, wherein the control module is further configured direct the energy source to emit the second treatment pulse to the first treatment area.

25. The system of Item 24, wherein the control module is further configured to redirect the energy source to emit the second treatment pulse at a second treatment area.

26. A method of operating an energy-based dermatological treatment system comprising:
aiming the energy-based dermatological treatment system at a first treatment area;
selectively emitting from an energy source a first treatment pulse to the first treatment area;
obtaining a first temperature measurement associated with the first treatment area;
generating a temperature map of the first treatment area based at least in part on the first temperature measurement; and
adjusting at least one parameter of a second treatment pulse to be emitted by the energy source based on the temperature map.

27. The method of Item 26, further including: selectively emitting from the energy source the second treatment pulse to the first treatment area.

28. The method of Item 26, further including
aiming the energy-based dermatological treatment system at a second treatment area; and
selectively emitting from the energy source the second treatment pulse to the second treatment area.

Accordingly, although the present disclosure has been provided in accordance with the implementations shown, one of ordinary skill in the art will readily recognize that there could be variations to the embodiments and those variations would be within the scope of the present disclosure. Therefore, many modifications may be made by one of ordinary skill in the art without departing from the scope of the appended claims.

What is claimed is:

1. An energy-based dermatological treatment system, comprising:
a temperature sensor for obtaining a first temperature measurement associated with a first treatment area;
a processing module for receiving the first temperature measurement and generate a temperature map based on the first temperature measurement;
a control module for setting a parameter of a first treatment pulse based on the temperature map; and
an energy source for delivering the first treatment pulse to the first treatment area,
wherein the temperature sensor is a contactless temperature sensor.

2. The system of claim 1, wherein the contactless temperature sensor includes an infrared temperature sensor.

3. The system of claim 1, wherein the parameter includes at least one of pulse intensity, pulse duration, and duty cycle.

4. The system of claim 1,
wherein the temperature sensor is further configured for obtaining a second temperature measurement associated with a second treatment area,
wherein the processing module is further configured for generating an updated temperature map based on the first and second temperature measurements,
wherein the control module is further configured for setting a parameter of a second treatment pulse based on the updated temperature map, and
wherein the energy source further configured for delivering the second treatment pulse to the second treatment area.

5. The system of claim 1, further comprising a cooling unit for convecting heat from the first treatment area.

6. The system of claim 5,
wherein the control module is operatively coupled with the cooling unit, and
wherein the control module is further configured for setting an operating parameter of the cooling unit based on the temperature map.

7. A method for operating an energy-based dermatological treatment system including an energy source for delivering treatment pulses, the method comprising:
selecting a first treatment area;
obtaining a first temperature measurement associated with the first treatment area;
generating a temperature map of the first treatment area based on the first temperature measurement;
setting a parameter of a first treatment pulse based on the temperature map; and
delivering the first treatment pulse to the first treatment area.

8. The method of claim 7, wherein the parameter includes at least one of pulse intensity, pulse duration, and duty cycle.

9. The method of claim 7, further comprising:
defining a lower threshold value for the parameter; and
generating an alert when the parameter is set below the lower threshold value.

10. The method of claim 7, further comprising:
defining an upper threshold value for the parameter; and
generating an alert when the parameter is set above the upper threshold value.

11. The method of claim 7, further comprising:
obtaining a second temperature measurement associated with the first treatment area;
generating an updated temperature map of the first treatment area based on the first and second temperature measurements;
adjusting a parameter of a second treatment pulse based on the updated temperature map; and
delivering the second treatment pulse to the first treatment area.

12. The method of claim 7, further comprising:
selecting a second treatment area;
obtaining a second temperature measurement associated with the second treatment area;
generating an updated temperature map of the first and second treatment areas based on the first and second temperature measurements;
adjusting a parameter of a second treatment pulse based on the second temperature map; and
delivering the second treatment pulse to the second treatment area.

13. The method of claim 12, wherein the first and second treatment pulses are delivered sequentially.

14. The method of claim 12, wherein the first and second treatment pulses are delivered substantially simultaneously.

15. The method of claim 12, further comprising generating an alert when the first and second treatment areas overlap.

16. The method of claim 7, wherein the energy-based dermatological treatment system further includes a cooling unit, the method further comprising cooling the first treatment area using the cooling unit.

17. A method for operating an energy-based dermatological treatment system including an energy source for delivering treatment pulses, the method comprising:
selecting a first treatment area;
delivering a first treatment pulse to the first treatment area;
obtaining a first temperature measurement associated with the first treatment area;
generating a temperature map of the first treatment area based on the first temperature measurement; and
setting a parameter of a second treatment pulse based on the temperature map.

18. The method of claim 17, wherein the energy-based dermatological treatment system further includes a cooling unit, the method further comprising cooling the first treatment area using the cooling unit prior to delivering the first treatment pulse.

19. The method of claim 17, further comprising delivering the second treatment pulse to the first treatment area.

20. The method of claim 19, wherein the energy-based dermatological treatment system further includes a cooling unit, the method further comprising cooling the first treatment area using the cooling unit during delivery of the first and second treatment pulses.

21. The method of claim 17, further comprising:
selecting a second treatment area adjacent to the first treatment area; and
delivering the second treatment pulse to the second treatment area.

22. The method of claim 20, wherein the energy-based dermatological treatment system further includes a cooling unit, the method further comprising cooling the first and second treatment areas using the cooling unit during delivery of the first and second treatment pulses.
